(19) United States
(12) Patent Application Publication
(10) Pub. No.: US 2004/0213835 A1
(43) Pub. Date: Oct. 28, 2004

(54) Method to reduce liposome-induced complement activation

(76) Inventors: Samuel Zalipsky, Redwood City, CA (US); Yechezkel Barenholz, Jerusalem (IL)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/789,489

(22) Filed: Feb. 26, 2004

Related U.S. Application Data
(60) Provisional application No. 60/451,362, filed on Feb. 28, 2003.
(60) Provisional application No. 60/524,176, filed on Nov. 21, 2003.

(51) Int. Cl. A61K 9/127

(52) U.S. Cl. 424/450

(57) ABSTRACT

A method for reducing complement activation upon in vivo administration of a liposome preparation containing an entrapped therapeutic agent is described. The method involves providing liposomes having a polyethylene-glycol-derived neutral lipopolymer.

This application claims the benefit of U.S. Provisional Application No. 60/451,362, filed Feb. 28, 2003 and of U.S. Provisional Application No. 60/524,176, filed Nov. 21, 2004, both of which are incorporated herein by reference in their entirety.

### FIELD OF THE INVENTION

The present invention relates to a method to reduce liposome-induced complement activation in vivo.

### BACKGROUND OF THE INVENTION

Liposomes are used for a variety of therapeutic purposes, particularly for carrying therapeutic agents to target cells by systemic administration of liposomal formulations of these agents. Liposome-drug formulations offer the potential of improved drug-delivery properties, such as controlled drug release. An extended circulation time is often needed for liposomes to reach the target region, cell or site from the site of injection. Therefore, when liposomes are administered systemically, it is desirable to coat the liposomes with a non-interacting agent, for example, a coating of hydrophilic polymer chains such as polyethylene glycol, to extend the blood circulation lifetime of the liposomes. Such surface-modified liposomes are commonly referred to as “long circulating” or “sterically stabilized” liposomes. The most common surface modification is attachment of PEG chains, typically having a molecular weight between 1000-5000, to about five mole percent of the lipids making up the liposomes. See, for example, Lasic, D. and Martin, F., Eds., “STEALTH LIPOSOMES”, CRC Press, Boca Raton, Fla., 1995, pp.108-100, and references therein. The pharmacokinetics exhibited by such liposomes are characterized by a dose-independent reduction in uptake of liposomes by the liver and spleen (via the mononuclear phagocyte system, or MPS) and significantly prolonged blood circulation time, as compared to non-surface-modified liposomes, which tend to be rapidly removed from the blood and to accumulate in the liver and spleen (ld.).

The most commonly used and commercially available PEG-substituted phospholipids are based on phosphatidylethanolamine, usually distearoyl phosphatidyl ethanolamine (DSPE), which is negatively charged at the polar head group. Negative surface charge in a liposome can be disadvantageous in some aspects, e.g. in interactions with cells (see e.g. Miller, C. M. et al., Biochemistry, 37:12875-12883 (1998)) and in delivery of cationic drugs, where leakage of the drug may occur (see e.g. Webb, M. S. et al., Biochim. Biophys. Acta, 1372:272-282 (1998)).

One recognized problem that results from in vivo administration of some liposome compositions in some individuals is induction of complement activation (Laverman, P. et al., Critical Reviews in Therapeutic Drug Carrier Systems, 18(6):551 (2001); Szebeni, J. et al., Am. J. Physiol Heart Circ. Physiol., 279:H1319 (2000); Szebeni, J. et al., Critical Reviews in Therapeutic Drug Carrier Systems, 15(1):57 (1998)). The complement system is the major effector of the humoral branch of the immune system and consists of nearly thirty serum and membrane proteins. Following initial activation, the various complement components interact in a highly regulated enzymatic cascade to generate reaction products that facilitate antigen clearance and generation of an inflammatory response. There are two pathways of complement activation: the classical pathway and the alternative pathway. The two pathways share a common terminal reaction sequence that generates a macromolecular membrane-aftack complex (MAC) which lyses a variety of cells, bacteria, and viruses (Kuby, Janis, IMMUNOLOGY, W.H. Freeman and Company, Chapter 14, 1997).

The complement reaction products amplify the initial antigen-antibody reaction and convert that reaction into a more effective defense. A variety of small, diffusible reaction products that are released during complement activation induce localized vasodilation and attract phagocytic cells chemotactically, leading to an inflammatory reaction. As antigen becomes coated with complement reaction products, it is more readily phagocytosed by phagocytic cells that bear receptors for these complement products (Kuby, Janis, IMMUNOLOGY, W.H. Freeman and Company, Chapter 14, 1997).

Complement activation has been reported to have a causal role in the cardiovascular distress caused by liposomal preparations administered in vivo, such as the commercially available preparations of pegylated liposomal doxorubicin (Doxil®, Caelyx®) and the pegylated liposome preparation HYNIC-PEG used in scintigraphic diagnosis of Crohn's colitis (Szebeni, J. et al., Am. J. Physiol Heart Circ. Physiol., 279:H1319 (2000); Szebeni, J. et al., Critical Reviews in Therapeutic Drug Carrier Systems, 15(1):57 (1998); Szebeni, J. et al., J. Liposome Res., 12(1&2):165 (2002)). Symptoms reported upon infusion of these preparations include cardiopulmonary distress, such as dyspnea, tachypnea, hypo- and/or hyper-tension, chest pain, back pain, flushing, headache, and chills (Szebeni, J. et al., Am. J. Physiol Heart Circ. Physiol., 279:H1319 (2000)).

Liposome-induced complement activation varies with a number of factors, and it has not yet been clarified which factors or combination of factors are the primary causitive agents. Liposome-induced complement activation appears to vary with lipid saturation, cholesterol content, the presence of charged phospholipids, and liposome size (Bradley, A. J., Archives of Biochem. and Biophys., 357(2):185 (1998)).

It would be desirable to provide a liposome preparation that reduces the complement activation response upon in vivo administration.

### SUMMARY OF THE INVENTION

In one aspect, the invention includes a method of reducing liposome-induced complement activation upon in vivo administration of liposomes containing an entrapped therapeutic agent. The method is comprised of providing liposomes that include a vesicle-forming lipid and between 1-10 mole percent, more preferably 1-5 mole percent, of a neutral lipopolymer having the formula:

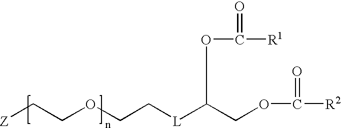

where each of R¹ and R² is an alkyl or alkenyl chain having between 8 and 24 carbon atoms; n=10-300, Z an inert end group selected from C₁-C₃ alkoxy, C₁-C₃ alkyl ether, n-methylamide, dimethylamide, methylcarbonate, dimethylcarbonate, carbamate, amide, n-methylacetamide, hydroxy, benzyloxy, carboxylic ester, and C₁-C₃ alkyl or aryl carbonate; and L is selected from the group consisting of (i) —X—(C═O)—Y—CH₂—, (ii) —X—(C═O)—, and (iii) —X—CH₂—, where X and Y are independently selected from oxygen, NH, and a direct bond, with the proviso that when L is —X—(C═O)—, X is not NH; and the remainder vesicle-forming lipids.

In one embodiment, X is oxygen and Y is nitrogen.

In another embodiment, L is a carbamate linkage, an ester linkage, or a carbonate linkage. In other embodiments, L is —O—(C═O)—NH—CH₂— (a carbamate linkage).

Z, in one embodiment, is hydroxy or methoxy.

The neutral lipopolymer, in preferred embodiments, in distearoyl (carbamate-linked) polyethylene glycol or methoxy-polyethelene glycol 1,2 distearoyl glycerol.

In another embodiment, each of R¹ and R² is an unbranched alkyl or alkenyl chain having between 8 and 24 carbon atoms. In a preferred embodiment, each of R¹ and R² is C₁₇H₃₅.

In yet another embodiment, n is between about 20 and about 115.

The therapeutic drug, in one embodiment, is a chemotherapeutic agent. Exemplary drugs include anthracycline antiobiotic, such as doxorubicin, daunorubicin, epirubicin, and idarubicin. Other exemplary drugs include platinum-containing compounds, such as cisplatin or a cisplatin analogue selected from the group consisting of carboplatin, ormaplatin, oxaliplatin, ((−)-(R)-2-aminomethylpyrrolidine (1,1-cyclobutane dicarboxylato))platinum, zeniplatin, enloplatin, lobaplatin, (SP-4-3(R)-1,1-cyclobutane-dicarboxylato(2-)-(2-methyl-1,4-butanediamine-N,N′))platinum, nedaplatin and bis-acetato-ammine-dichloro-cyclohexylamine-platinum(IV).

These and other objects and features of the invention will be more fully appreciated when the following detailed description of the invention is read in conjunction with the accompanying drawings.

### BRIEF DESCRIPTION OF THE DRAWINGS

FIG. 1 shows a synthetic scheme for the preparation of a carbamate-linked uncharged lipopolymer, referred to herein as PEG-DS;

FIGS. 2A-2D show synthetic schemes for preparation of ether-, ester-, amide-, and keto-linked uncharged lipopolymers;

FIGS. 3A-3C are graphs showing the biodistribution of HSPC/Chol liposomes containing 3 mole % PEG-DS (FIG. 3A); 5 mole % PEG-DSPE (FIG. 3B); or 5 mole % PEG-DS (FIG. 3C), in the blood, liver, and spleen;

FIG. 4 is a graph showing the retention in the blood of hydrogenated soy phosphatidylcholine liposomes containing no PEG lipid (crosses), 5 mole % PEG-DSPE (triangles), or 5 mole % PEG-DS (circles);

FIG. 5 shows a synthetic scheme for preparation of a neutral-zwitterionic mPEG-lipid conjugate derived from a natural phospholipids, such as phosphatidylethanolamine or phosphatidylglycerol; and

FIG. 6 shows the induction of complement activation in human serum in vitro, as measured by SC5b-9 induction for Preparation nos. 1, 3, 4, 5, 6, 8, 9, and 10, expressed as a percentage of SC5b-9 induction via phosphate buffered saline (PBS).

### DETAILED DESCRIPTION OF THE INVENTION

### I. Definitions

As used herein, a “neutral” lipopolymer is one that is uncharged, having no net charge, i.e., if any, there is an equal number of positive and negative charges.

“Vesicle-forming lipids” refers to amphipathic lipids which have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. The vesicle-forming lipids of this type typically include one or two hydrophobic acyl hydrocarbon chains or a steroid group, and may contain a chemically reactive group, such as an amine, acid, ester, aldehyde or alcohol, at the polar head group. Included in this class are the phospholipids, such as phosphatidyl choline (PC), phosphatidyl ethanolamine (PE), phosphatidic acid (PA), phosphatidyl inositol (PI), and sphingomyelin (SM), where the two hydrocarbon chains are typically between about 14-22 carbon atoms in length, and have varying degrees of unsaturation. Other vesicle-forming lipids include glycolipids, such as cerebrosides and gangliosides, and sterols, such as cholesterol. For the compositions described herein, phospholipids, such as PC and PE, cholesterol, and the neutral lipopolymers described herein are preferred components.

“Alkyl” refers to a fully saturated monovalent radical containing carbon and hydrogen, and which may be branched or a straight chain. Examples of alkyl groups are methyl, ethyl, n-butyl, t-butyl, n-heptyl, and isopropyl. “Lower alkyl” refers to an alkyl radical of one to six carbon atoms, as exemplified by methyl, ethyl, n-butyl, i-butyl, t-butyl, isoamyl, n-pentyl, and isopentyl.

“Alkenyl” refers to monovalent radical containing carbon and hydrogen, which may be branched or a straight chain, and which contains one or more double bonds.

Abbreviations: PEG: polyethylene glycol; mPEG: methoxy-terminated polyethylene glycol; Chol: cholesterol; PC: phosphatidyl choline; PHPC: partially hydrogenated phosphatidyl choline; PHEPC: partially hydrogenated egg phosphatidyl choline; HSPC: hydrogenated soy phosphatidyl choline; DSPE: distearoyl phosphatidyl ethanolamine; DSP or PEG-DS: distearoyl (carbamate-linked) PEG; APD: 1-amino-2,3-propanediol; DTPA: diethylenetetramine pentaacetic acid; Bn: benzyl.

### II. Method of Reducing Complement Activation

In one aspect, the invention provides a method for reducing induction of complement activation upon in vivo administration of a liposome preparation to a human. As will be described below, the method includes providing a liposome preparation that includes a neutral lipopolymer, or in an alternative embodiment, a neutral-zwitterionic lipopolymer. The invention also includes a liposome composition comprising a neutral lipopolymer, or in an alternative embodiment, a neutral-zwitterionic lipopolymer for use in reducing induction of complement activation upon in vivo administration of the liposome preparation. The invention further contemplates use of the liposome composition for preparation of a medicament for use in reducing complement activation in a subject.

A. Liposome Preparation

The PEG-substituted neutral lipopolymers of the invention have the structure shown below:

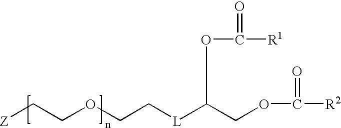

where

each of R¹ and R² is an alkyl or alkenyl chain having between 8 and 24 carbon atoms;

n is between about 10 and about 300,

Z is an inert end group, selected from the group consisting of C₁-C₃ alkoxy, C₁-C₃ alkyl ether, n-methylamide, dimethylamide, methylcarbonate, dimethylcarbonate, carbamate, amide, n-methylacetamide, hydroxy, benzyloxy, carboxylic ester, and C₁-C₃ alkyl or aryl carbonate; and

L is selected from the group consisting of (i) —X—(C═O)—Y—CH₂—, (ii) —X—(C═O)—, and (iii) —X—CH₂—, where X and Y are independently selected from oxygen, NH, and a direct bond.

The end group, Z, is selected for minimal interaction with in vivo components that induce complement activation. Z preferably is a moiety that acts as a hydrogen bond acceptor that binds water and is incapable of serving as a hydrogen bond donor. Exemplary inert moieties suitable for Z include C₁-C₅ alkoxy, more preferably C₁-C₃ alkoxy, C₁-C₅ alkyl ether, more preferably C₁-C₃ alkyl ether, n-methylamide, dimethylamide, methylcarbonate, dimethylcarbonate, carbamate, amide, n-methylacetamide, hydroxy, benzyloxy, carboxylic ester, and C₁-C₃ alkyl or aryl carbonates. Preferred Z moieties include methoxy, ethoxy, and n-methylacetamide.

The lipopolymers include a neutral linkage (L) in place of the charged phosphate linkage of PEG-phospholipids, such as PEG-DSPE, which are frequently employed in sterically stabilized liposomes. L can contain charged moieties provided the net charge is zero, e.g, L is zwitterionic. The neutral linkage can be, for example, a carbamate, an ester, an amide, a carbonate, a urea, an amine, an ether, sulfur, or sulfur dioxide. Hydrolyzable or otherwise cleavable linkages, such as carbonates and esters, are preferred in applications in which it is desirable to remove the PEG chains after a given circulation time in vivo. This feature can be useful in releasing drug or facilitating uptake into cells after the liposome has reached its target (Martin, F. J. et al., U.S. Pat. No. 5,891,468 (1999); Zalipsky, S. et al., PCT Publication No. WO 98/18813 (1998)).

The PEG group attached to the linking group preferably has a molecular weight between about 1000 and 15000; that is, where n is between about 20 and about 340. More preferably, the molecular weight is between about 1000 and 12000 (n=about 20-275), and most preferably between about 1000 and 5000 (n=about 20-115). The R¹ and R² groups are preferably between 16-20 carbons in length, with R¹=R²=C₁₇H₃₅ (such that COOR is a stearyl group) being particularly preferred.

As stated above, the incorporation of an uncharged lipid into liposomes can present advantages such as reduced leakage of encapsulated amphipathic weak basic or acidic drugs. Another advantage is greater flexibility in modulating interactions of the liposomal surface with target cells and with the RES (Miller, C. M. etal., Biochemistry, 37:12875-12883 (1998)). PEG-substituted synthetic ceramides have been used as uncharged components of sterically stabilized liposomes (Webb, M. S. et al., Biochim. Biophys. Acta, 1372:272-282 (1998)); however, these molecules are complex and expensive to prepare, and they generally do not pack into the phospholipid bilayer as well as diacyl glycerophospholipids.

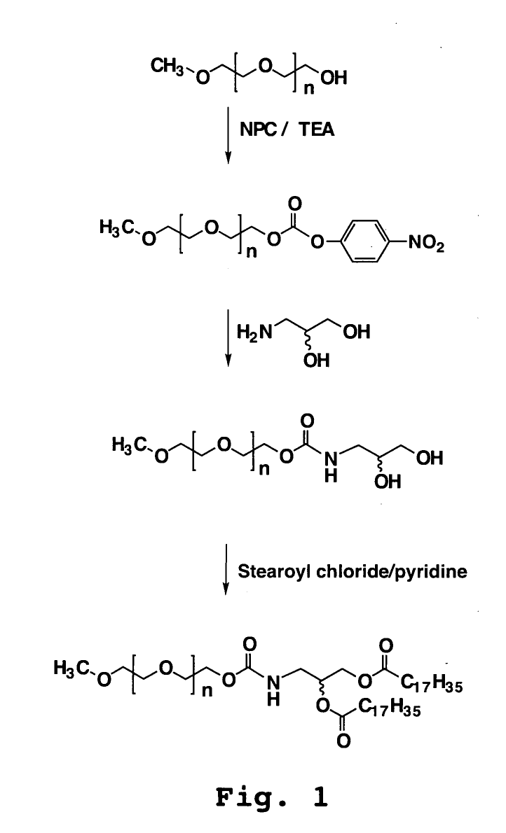

The lipopolymers can be prepared using standard synthetic methods. For example, the carbamate-linked compound (L=—O—(C═O)—NH—CH₂—) is prepared, as shown in FIG. 1, by reacting the terminal hydroxyl of mPEG (methoxy-PEG) with p-nitrophenyl chloroformate, to give the p-nitrophenyl carbonate, which is then reacted with 1-amino-2,3-propanediol to give the intermediate carbamate. The hydroxyl groups of the vicinal diol moiety are then acylated to give the final product. A similar route, using glycerol in place of 1-amino-2,3-propanediol, can be used to produce a carbonate-linked product (L=—O—(C═O)—O—CH₂—). Preparation of carbamate-linked distearoyl and diecosanoyl lipopolymers is described in Examples 1 and 2.

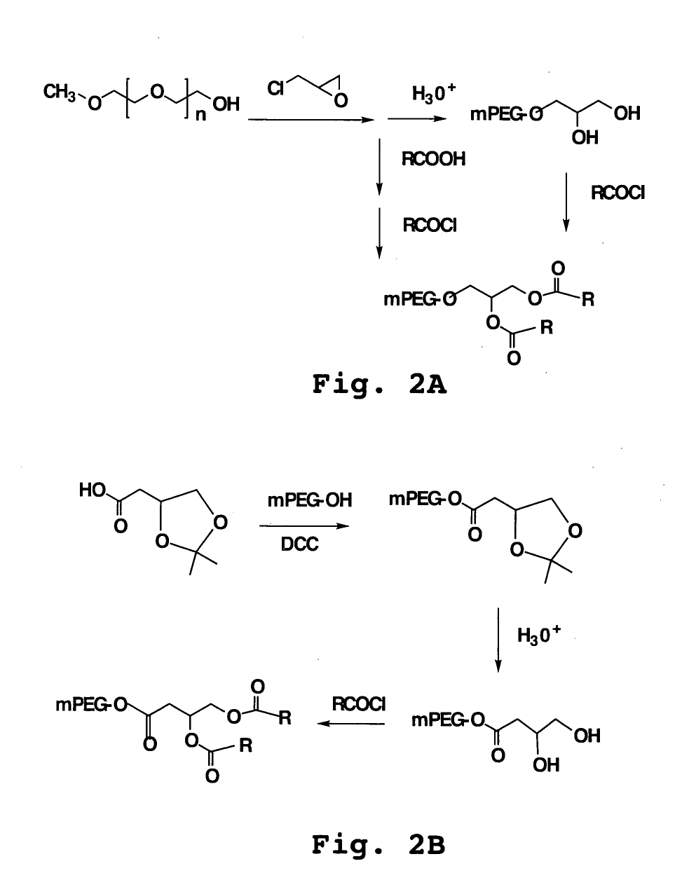

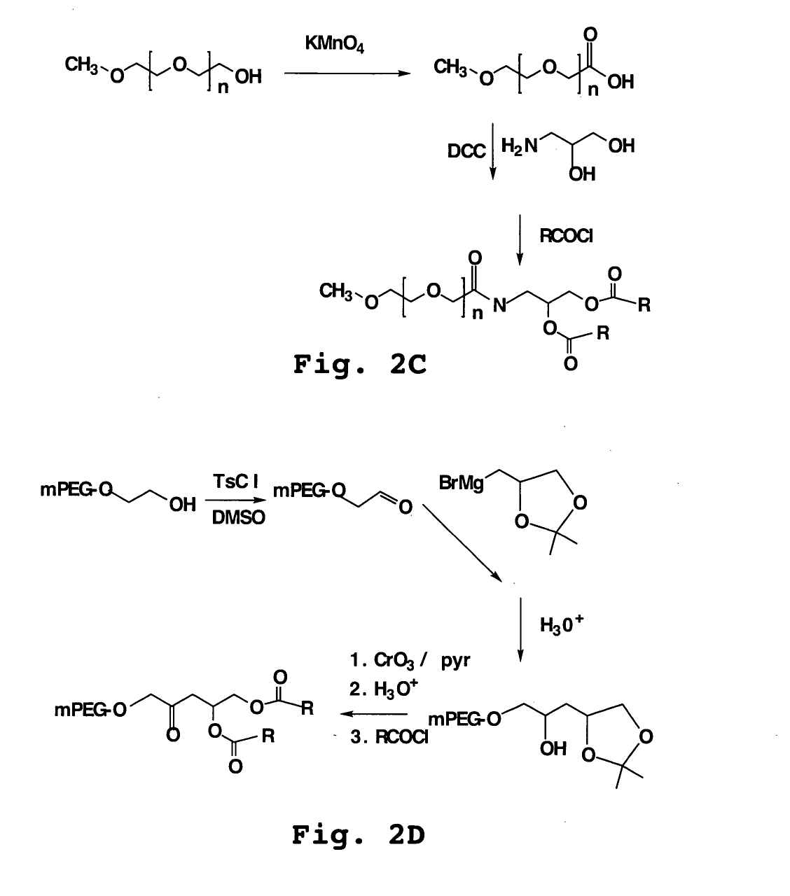

As shown in FIG. 2A, an ether-linked lipopolymer (L=—O—CH₂—) is readily prepared by reacting the terminal hydroxyl of mPEG-OH with glycidyl chloride (e.g., epichlorohydrine), hydrolyzing the resulting epoxide, and acylating the resulting diol. Ester-linked lipopolymers (L=—O—(C═O)— or —O—(C═O)—CH₂—) can be prepared, for example, as shown in FIG. 2B, by reacting mPEG-OH with an activated derivative of glyceric acid acetonide (2,2-dimethyl-1,3-dioxolane-4-carboxylic acid) or the four-carbon homolog, 2,2-dimethyl-1,3-dioxolane-4-acetic acid, as shown. The diol is then deprotected and acylated.

Corresponding reactions using mPEG-NH₂, prepared e.g. by the method of Zalipsky, S. et al. (Eur. Polym. J., 19:1177-1183 (1983)) in place of mPEG-OH, may be used to prepare lipopolymers having amide, urea or amine linkages (i.e., where L=—NH—(C═O)—NH—, —NH—(C═O)—CH₂—, —NH—(C═O)—NH—CH₂—, or —NH—CH₂—).

Compounds in which L is —X—(C═O)—, where X is O or NH, can be prepared by reaction of an activated carboxyl-terminated PEG (prepared by oxidation of hydroxyl-terminated PEG and activation of the carboxyl group by, for example, conversion to the nitrophenyl ester or reaction with DCC) with 1,2,3-propanetriol or 1-amino-2,3-propanediol, respectively (FIG. 2C). A keto-linked compound (i.e. where X is a direct bond) may be prepared by condensation of aldehyde terminated PEG (prepared by mild oxidation of hydroxyl-terminated PEG) with, for example, the Grignard reagent of 1-bromo-2,3-propanediol acetonide (FIG. 2D), followed by oxidation to the ketone, under non-acidic conditions, and hydrolysis of the acetonide to the diol. In each case, the diol is then acylated as usual.

The terminus of the PEG oligomer not linked to the glycerol moiety (α terminus; group Z above) is typically hydroxy or methoxy, but may be functionalized, according to methods known in the art, to facilitate attachment of various molecules to the neutral lipopolymer, for use in targeting the liposomes to a particular cell or tissue type or otherwise facilitating drug delivery. Molecules to be attached may include, for example, peptides, saccharides, antibodies, or vitamins. Examples 2-3 below describe steps in the preparation of α-functionalized lipopolymers following routes similar to those described above, but starting with commercially available PEG oligomers in which the α terminus is substituted with a group, such as t-butyl ether or benzyl ether, which is readily converted to hydroxyl after synthesis of the lipid portion of the molecule. This terminus is then activated, in this case by conversion to a p-nitrophenylcarbonate.

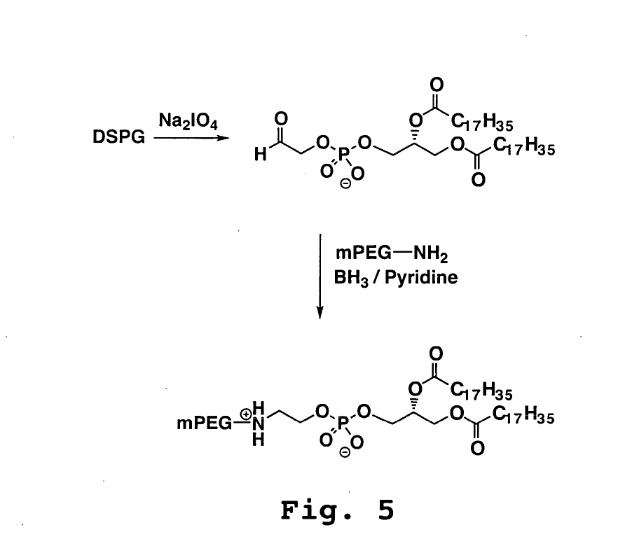

Another exemplary neutral lipopolymer is illustrated in FIG. 5. Synthesis of a neutral-zwitterionic polymer-lipid is exemplified using the polymer PEG and the lipid DSPG. It will be appreciated that other hydrophilic polymers and other lipids could also be used; for example, reductive alkylation of phosphatidyethanolamine with mPEG aldehyde. In brief and as described in more detail in Example 4, DSPG was oxidized by treating with sodium periodate and then reacted with mPEG-NH2 in the presence of borane-pyridine to form a neutral-zwitterionic mPEG-DSPE polymer. The zwitterionic lipopolymer has a net neutral charge at physiological pH. It will for liposomal bilayers that are neutral, eliminating undesirable charges in the liposomal particle.

B. Liposome Pharmacokinetics

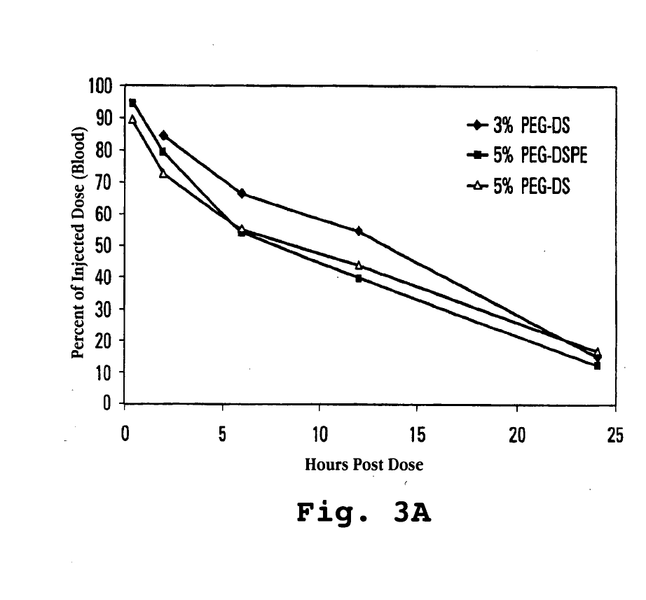

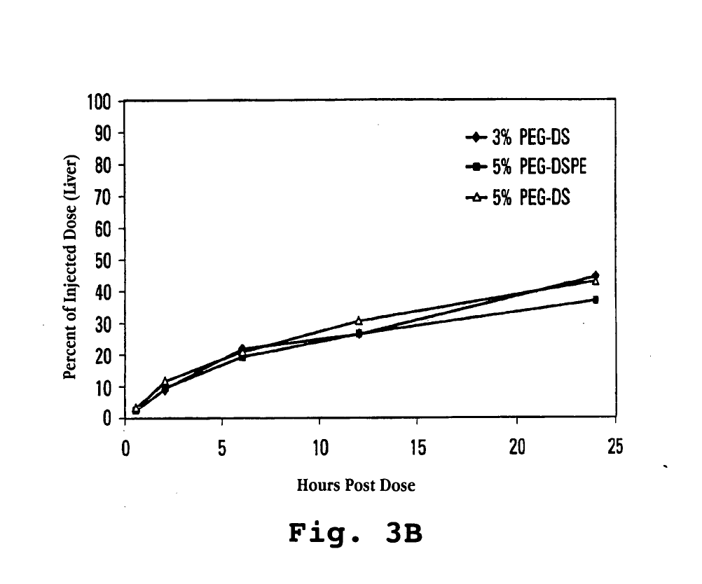

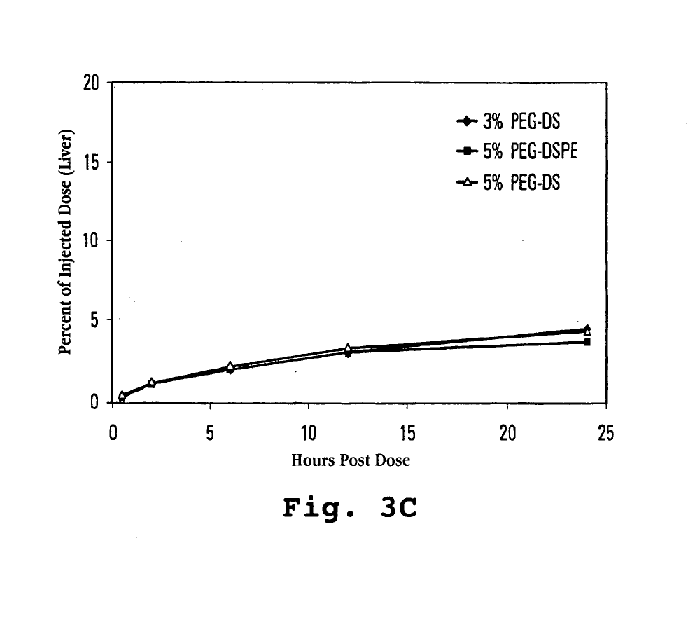

Long-circulating liposomes are formed by incorporating 1-10 mole %, more preferably 1-5 mole %, and more preferably 3-10 mole %, of a neutral lipopolymer, or a neutral-zwitterionic polymer, into liposomes composed of vesicle-forming lipids. To illustrate, liposomes incorporating 3 to 5 mole % of either mPEG₂₀₀₀-DSPE (distearoyl phosphatidyl ethanolamine) or carbamate linked lipopolymer mPEG₂₀₀₀-DS were prepared as described in Example 5. The balance of the lipids consisted of HSPC and cholesterol in a 1.5:1 mole ratio. The liposomes were loaded with the marker ¹²⁵I-tyraminylinulin. A sample of each preparation was injected into the tail vein of mice, and the tissue distribution was determined at various time points, as described in Example 5. Levels present in the blood, liver and spleen are shown in Tables 1A-1C and graphically in FIGS. 3A-3C. As the data shows, the pharmacokinetics of the PEG-DS-containing liposomes were very similar to those of the liposomes containing PEG-DSPE.

TABLE 1A
Liposome Distribution in Blood

| Time | % of Injected Dose | | |
|---|---|---|---|
| Point | A | B | C |
| 30 min | — | 94.8 ± 3.99 | 89.7 ± 6.94 |
| 2 h | 85.1 ± 1.99 | 79.8 ± 3.42 | 73.0 ± 17.4 |
| 6 h | 67.1 ± 6.25 | 54.5 ± 3.05 | 55.3 ± 2.51 |
| 12 h | 54.9 ± 6.04 | 39.7 ± 2.52 | 44.4 ± 2.52 |
| 24 h | 14.8 ± 2.81 | 12.4 ± 2.34 | 16.6 ± 2.38 |

TABLE 1B
Liposome Distribution in Liver

| Time | % of Injected Dose | | |
|---|---|---|---|
| Point | A | B | C |
| 30 min | — | 2.27 ± 0.13 | 3.14 ± 0.95 |
| 2 h | 8.76 ± 2.01 | 9.42 ± 1.24 | 11.7 ± 1.74 |
| 6 h | 21.7 ± 2.55 | 19.3 ± 1.37 | 20.8 ± 0.86 |
| 12 h | 26.6 ± 0.51 | 26.4 ± 1.99 | 30.4 ± 1.28 |
| 24 h | 43.9 ± 2.7 | 36.6 ± 2.25 | 42.6 ± 0.48 |

TABLE 1C
Liposome Distribution in Spleen

| Time | % of Injected Dose | | |
|---|---|---|---|
| Point | A | B | C |
| 30 min | — | 0.09 ± 0.06 | 0.23 ± 0.08 |
| 2 h | 0.96 ± 0.16 | 0.99 ± 0.09 | 1.08 ± 0.09 |
| 6 h | 1.94 ± 0.07 | 1.96 ± 0.29 | 2.12 ± 0.13 |
| 12 h | 3.15 ± 0.31 | 3.13 ± 0.12 | 3.35 ± 0.22 |
| 24 h | 4.69 ± 0.37 | 3.91 ± 0.31 | 4.56 ± 0.29 |

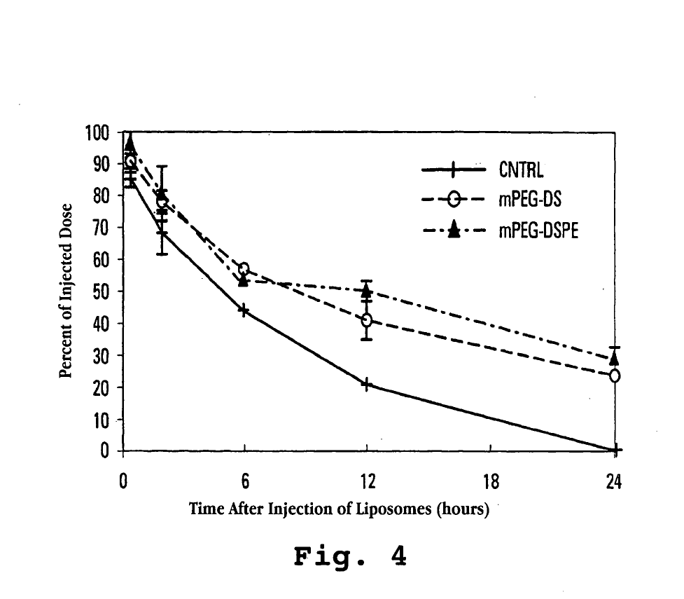

A similar study compared the performance of both PEG lipids against a control formulation, containing no PEG lipid. FIG. 4 shows the retention in the blood of 2:1 HSPC liposomes containing no PEG lipid (crosses), 5 mole % PEG₂₀₀₀-DSPE (triangles), or 5 mole % PEG₂₀₀₀-DS (circles).

Further studies were done using liposomes containing mPEG₂₀₀₀-DS:PHPC: Chol in a 5:55:40 molar ratio. The liposomes were labeled by incorporation of an indium-DTPA complex. Percent of injected dose was determined in the blood and in various tissues at 24 hours. The results are shown in Tables 2A-2C. Again, the liposomes showed typical long-circulating pharmacokinetics, with an average retention of >70% of the injected dose after 4 hours, and >30% after 24 hours.

TABLE 2A
Percent of Injected Dose of Indium in Blood

| Animal # | 0.0 hrs | 0.5 hrs | 1.0 hrs | 2.0 hrs | 4.0 hrs | 24 hrs |
|---|---|---|---|---|---|---|
| Rat 1 | 103.7 | 91.2 | 82.5 | 73.8 | 72.0 | 33.1 |
| Rat 2 | 97.7 | 87.7 | 79.4 | 78.7 | 74.4 | 30.7 |
| Rat 3 | 95.1 | 83.1 | 77.8 | 68.6 | 64.4 | 29.8 |
| Rat 4 | 91.9 | 85.4 | 78.5 | 75.6 | 72.6 | 33.2 |
| Average | 97.1 | 86.8 | 79.6 | 74.2 | 70.9 | 31.7 |
| Std. Dev. | 5.0 | 3.4 | 2.1 | 4.2 | 4.4 | 1.7 |

TABLE 2B
Percent of Injected Dose in Tissues at 24 Hours

| Tissue | Rat #1 | Rat #2 | Rat #3 | Rat #4 | Average | Std. Dev. |
|---|---|---|---|---|---|---|
| Liver | 7.5 | 6.9 | 6.7 | 7.2 | 7.1 | 0.3 |
| Spleen | 4.9 | 5.4 | 5.6 | 4.8 | 5.2 | 0.4 |
| Heart | 0.4 | 0.5 | 0.5 | 0.6 | 0.5 | 0.1 |
| Kidneys | 1.2 | 1.2 | 1.0 | 1.2 | 1.1 | 0.1 |
| Lung | 0.7 | 0.7 | 0.7 | 0.8 | 0.7 | 0.1 |
| Skin | 0.1 | 0.3 | 0.2 | 0.2 | 0.2 | 0.1 |
| Bone | 0.3 | 0.2 | 0.2 | 0.2 | 0.2 | 0.2 |
| Muscle | 0.1 | 0.1 | 0.1 | 0.2 | 0.1 | 0.4 |
| Urine | 11.2 | 13.4 | 5.7 | 12.3 | 10.7 | 3.4 |

TABLE 2C
Percent of Injected Dose Per Gram in Tissues at 24 Hours

| Tissue | Rat #1 | Rat #2 | Rat #3 | Rat #4 | Average | Std. Dev. |
|---|---|---|---|---|---|---|
| Liver | 0.7 | 0.7 | 0.7 | 0.7 | 0.7 | 0.3 |
| Spleen | 7.3 | 6.9 | 8.2 | 5.9 | 7.1 | 0.9 |
| Heart | 0.5 | 0.5 | 0.5 | 0.5 | 0.5 | 0.4 |
| Kidneys | 0.6 | 0.6 | 0.5 | 0.6 | 0.6 | 0.6 |
| Lung | 0.6 | 0.5 | 0.5 | 0.6 | 0.5 | 0.6 |
| Skin | 0.1 | 0.1 | 0.1 | 0.1 | 0.1 | 0.1 |
| Bone | 0.4 | 0.4 | 0.4 | 0.4 | 0.4 | 0.3 |
| Muscle | 0.1 | 0.1 | 0.1 | 0.1 | 0.1 | 0.2 |
| Urine* | 0.6 | 0.6 | 0.3 | 0.8 | 0.6 | 0.2 |

*Percent of injected dose per mL.

Liposomes containing 5 mole % mPEG₂₀₀₀-DS or mPEG₂₀₀₀-DSPE and the remainder PHEPC were compared with respect to percent remaining in the blood up to 24 hours post administration. As shown in FIG. 4, the pharmacokinetics were virtually identical, with approximately 40% retention after 24 hours.

C. Measurement of Complement Activation In vitro

To evaluate the effect of liposome preparations comprised of the neutral lipopolymer on induction of complement activation, twelve liposome preparations and two micellar preparations were prepared, as described in Example 6. Table 3 in Example 6 details the lipid composition of the preparations. In brief and with reference to Table 4, the preparations included:

PREPARATION NOS. 1, 2, 3: two drug-loaded liposomes of identical lipid composition, differing only in the entrapped drug, doxorucibin (Doxil®) and cisplatin (preparation numbers 1 and 2) and a preparation of identical lipid composition but with no entrapped therapeutic agent, i.e., placebo (preparation no. 3);

PREPARATION NO. 4: the effect of amount of PEG₂₀₀₀-DSPE on induction of complement activation was evaluated by comparing a preparation with 0.6 mole % PEG₂₀₀₀-DSPE with preparation no. 3 which was identical but for a higher (4.5 mole %) amount of PEG₂₀₀₀-DSPE;

PREPARATION NOS. 5, 6, 7: the effect of the negative charge of the PEG-DSPE was studied by comparing the preparation no. 3 (placebo to Doxil® and cisplatin liposome preparation nos. 1 and 2) with liposome preparations in which the negatively-charged PEG₂₀₀₀-DSPE was removed (preparation no. 6) replaced with two neutral lipopolymer: PEG₂₀₀₀-DS (preparation no. 7) and PEG₂₀₀₀-DSG (preparation no. 6; DSG=distearoyl glycerol; see FIG. 2A structure of mPEG-DSG);

PREPARATION NOS. 8, 9: the effect of the size of the PEG moiety on induction of complement activation was studied by comparing liposomes having negatively charged PEG-DSPE with different PEG molecular weights of 350 Daltons (preparation no. 8), 2000 Daltons (preparation no. 3), and 12,000 Daltons (preparation no. 9);

PREPARATION NO. 10: liposomes having a negative charge introduced through a liposome-forming phospholipid hydrogenated soy phosphatidyl glycerol (HSPG) were prepared for comparison with liposomes in which the negative charge was introduced through the micelle-forming lipopolymer PEG₂₀₀₀-DSPE, which has a large headgroup (preparation no. 3);

PREPARATION NOS. 11, 12: as a liposome-positive control, liposomes of large particle size and composed of DMPC/chol/DMPG with cholesterol mole fractions of 50% (preparation no. 11) and 71% (preparation no. 12), as these preparations are highly potent in activating the complement system, including complement-dependent cardiopulmonary distress in pigs;

PREPARATION NOS. 13, 14: to determine whether PEG₂₀₀₀-DSPE without other lipids induces complement activation, micelles of PEG₂₀₀₀-DSPE (preparation no. 13) and PEG₂₀₀₀-DS (preparation no. 14) were prepared.

A comparison of liposome preparation no. 10 with liposome preparation no. 3 provided a study of the difference between an exposed negative charge to a hidden negative charge, since liposomes having a negative charge introduced through the liposome-forming phospholipid HSPG have an exposed negative charge, whereas liposomes in which the negative charge was introduced through the lipopolymer PEG₂₀₀₀-DSPE have a negative charged shielded by the PEG chain.

Table 4 summarizes the liposome and micellar preparations and shows the size, surface charge (Ψ₀), and zeta potential.

TABLE 4
Characteristics of the Liposome Compositions

| | | Surface | Zeta |
|---|---|---|---|
| | Particle Size² | Potential² | Potential² |
| Formulation Number and Name¹ | (nm) | (mV) | (mV) |
| 1 - Doxil ® | 108 | — | −13.3 |
| 2 - cisplatin liposomes | 116 | −14.3 | −9.8 |
| 3 - Doxil ® placebo | 124 | −52 | −10.1 |
| 4 - 0.6% PEG₂₀₀₀-DSPE | 121 | −2.9 | −10.3 |
| 5 - HSPC/Chol | 135 | 0 | −4.6 |
| 6 - PEG-DS | 111 | −12.3 | −0.79 |
| 7 - EPC/PEG-DSG | 70 | — | 0.7 |
| 8 - PEG₃₅₀-DSPE | 127 | — | — |
| 9 - PEG₁₂₀₀₀-DSPE | 128 | — | — |
| 10 - HSPG | 135 | −81.34 | −52.5 |
| 11 - Low-Chol | >1000 | — | — |
| 12 - High-Chol | >1000 | — | — |
| 13 - PEG₂₀₀₀-DSPE micelles | 25 | −141 | −9.0 |
| 14 - PEG₂₀₀₀-DS micelles | 25 | −19 | −1.3 |

¹see Table 3 in Example 6 below for details of lipid composition
²see Example 6 for methodologies

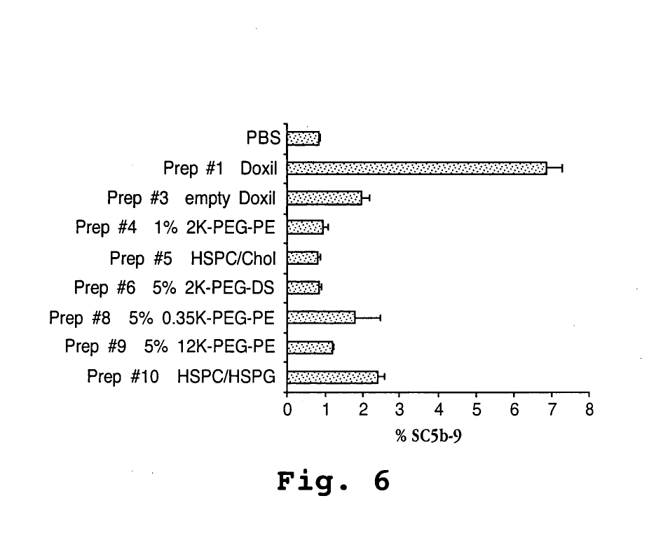

As described in Example 6, in vitro induction of complement activation was determined by measuring the formation of S-protein-bound C terminal complex (SC5b-9) as marker of complement activation upon incubation of human serum with the various liposome preparations. In a typical study, a liposome preparation was mixed with serum and incubated at 37° C. for about 30 minutes. The reaction was stopped, and the quantity of SC5b-9 was determined by an enzyme-linked immunosorbent assay. The results for Preparation Nos. 1, 3, 4, 5, 6, 8, 9, and 10 are shown in FIG. 6.

FIG. 6 shows the SC5b-9 induction, as a percent of the baseline SC5b-9 induction for cells incubated with phosphate buffered saline, for the indicated liposomal preparations. Liposome preparations 5 and 6 are neutral in charge (preparation no. 6 includes the neutral lipopolymer PEG-DS and preparation no. 5 is composed of the neutral lipids HSPC/Chol). These neutral preparations caused no measurable change in SC5b-9 formation. Preparation no. 5 containing 0.6% PEG₂₀₀₀-DSPE also invoked little complement activation. However, all the other liposome preparations caused a significant elevation of SC5b-9 relative to the PBS control. The “Doxil® placebo” preparation no. 3 and the negatively charged HSPG-containing liposome preparation no. 10 caused moderate, approximately 2-fold rise in SC5b-9 formation, the Doxil® preparation no. 1 caused a very strong, 7-fold increase of SC5b-9. These data suggest that the negative electric charge and, particularly, doxorubicin in Doxil®, are contributing factors to complement activation. This finding was confirmed by the fact that liposome preparation no. 2, the cisplatin-loaded liposomes having the same lipid composition and size of the Doxile liposome preparation no. 1 caused no or minor complement activation (data not shown). When HSPC was replaced by EPC as in preparation no. 7 (relative to preparation no. 6), a moderate but significant complement activation in ⅔ tested sera resulted.

The complement activating effects of preparation no. 13 (PEG₂₀₀₀-PE micelles) was evaluated by adding the micelles at increasing concentrations to human sera. Micelles caused no significant rise of SC5b-9 in either sera under conditions when Doxil® (preparation no. 1) caused significant activation (data not shown). In fact, micelles added up to 10-times higher concentration than Doxil® preparation no. 1 caused to complement activation. Thus, the spatial arrangement of complement binding sites on bilayer membranes may be an additional critical factor in liposome-induced complement activation.

D. Measurement of Complement Activation In vivo

Complement activation induced by the liposome preparations described above was evaluated in vivo by administering the preparations to pigs, as described in Example 7. For a unified quantification of multiple physiological changes underlying liposome-induced hypersensitivity (HSR) in pigs, a scoring system that qualifies these reactions from grade I to IV was developed. The scoring system is detailed in Example 7 and assigns grades I, II, III, and IV to physiologic responses of no reaction, moderate, severe, and lethal reactions, respectively. The dose dependence, frequency and grade of cardiopulmonary response of pigs to different liposomes is summarized in Table 5.

TABLE 5
Cardiopulmonary Response of Pigs to Different Liposomes

| | Bolus Dose | | | | | | |
|---|---|---|---|---|---|---|---|
| | (nmole phospholipid/kg) | | | | Reaction | | |
| Preparation No. | 5-30 | 30-150 | 150-1000 | 1000-10⁴ | Frequency (%) | Grade¹ | n |
| 1 - Doxil ® | | 1 | | | 93 | 0 | 1/14 |
| | | 1 | | | | II | 1/14 |
| | | 3 | | | | III | 3/14 |
| | 6 | 3 | | | | IV | 9/14 |
| 2 - cisplatin | | 2 | | | 0 | 0 | 2/2 |
| 3 - Placebo Doxil ® | 1 | | 1 | | 67 | 0 | 2/6 |
| | | 1 | | | | II | 1/6 |
| | | 3 | | | | IV | 3/6 |
| 4 - PEG₂₀₀₀-PE | | | 1 | | 0 | 0 | |
| 5 - HSPC/Chol | 1 | | 4 | 1 | 0 | 0 | 6/6 |
| 6 - PEG₂₀₀₀-DS | | | | 1 | 75 | 0 | 1/4 |
| | | | 3 | | | I | 3/4 |
| 7 - EPC/PEG-DSG | 1 | | | | 100 | IV | 1/1 |
| 8 - PEG₃₅₀-PE | | 2 | | | 33 | 0 | 2/3 |
| | | 1 | | | | II | 1/3 |
| 9 - PEG₂₀₀₀-PE | | 1 | | | 100 | II | 1/3 |
| | | 2 | | | | III | 2/3 |
| 10 - HSPG | | 3 | | | 100 | II | 3/5 |
| | | | 2 | | | IV | 2/5 |
| 11 - Low-chol | 40 | | | | 100 | III | 35/40 |
| | | | | | | IV | 8/40 |
| 12 - High-chol | 22 | | | | 100 | IV | 22/22 |
| 13 - PEG-DSPE micelles | 2 | | | | 0 | 0 | 2/2 |
| 14 - PEG-DS micelles | 2 | | | | 0 | 0 | 2/2 |
| 15 - EPC/Chol/EPG | 2 | | | | 100 | III | 2/4 |
| | 2 | | | | | IV | 2/4 |

¹For definitions of grades see Example 7
²Lipid compositions of the preparations are given in Table 3 in Example 6.

Consistent with the observation that preparation no. 1 (Doxil®), as well as negatively charged PE-containing liposomes (preparation nos. 8, 9, 10), were potent complement activators in human serum in vitro (FIG. 6), these same liposomes were the most potent inducers of cardiopulmonary distress in pigs with 3-150 nmole phospholipid/kg causing severe to lethal reactions in >90% of the tests. The minimum dose of preparation no. 1 (Doxil®) causing hypersensitivity reaction was 50 μL from the original vial containing 2 mg/mL doxorubicin and 12.8 mg/mL phospholipid, corresponding to 1/400 to 1/1000 part of the human therapeutic dose that approximately reaches the blood in the initial 15-30 seconds of infusion. The dose dependence of Doxil®'s reactogenicity in humans and in pigs was practically identical.

In further agreement with in vitro complement activation, equivalent doses of preparation no. 3 (placebo Doxil®) also caused hypersensitivity reactions in pigs but at a lower rate (67%), while preparation no. 4 (PEG₂₀₀₀-DSPE), preparation no. 8 (PEG₃₅₀-DSPE), and preparation no. 6 (PEG₂₀₀₀-DS) and preparation nos. 13, 14 (PEG₂₀₀₀ micelles) caused no or mild reactions even at higher doses. The only apparent divergence between in vitro complement activation and porcine hypersensitivity reactions was the two severe reactions out of three tests to preparation no. 9 (PEG₁₂₀₀₀-DSPE liposomes), which caused no or minor complement activation in human sera.

Both preparation no. 6 and preparation no. 7 were prepared from neutral lipids. Preparation no. 6 was formed of HSPC, cholesterol, and PEG-DS. Preparation no. 7 was formed of EPC and PEG-DSG, a commercially available neutral lipopolymer (see Example 6). However, the in vivo response of the two preparations differed in that preparation no. 7 resulted in induction complement activation sufficiently severe to cause death in the test animal. In contrast, the response to preparation no. 6 was a Grade I or minimal response in three of four test animals, and was a Grade 0 (no response) in one test animal. This results suggests that not all neutral lipopolymers are capable of reducing the induction of complement activation caused upon in vivo administration of a liposome preparation.

In another study conducted in pigs, four liposome preparations were prepared, as described in Example 8. The lipid composition and characteristics of the four preparations are shown in Table 6.

TABLE 6
Liposome Preparations for in vivo Evaluation of Induction
of Complement Activation

| Lipid composition | | | | | | Measured | |
|---|---|---|---|---|---|---|---|
| mg (μmol) | | | | | | phospholipoid | |
| Prep | | | | | | concentration | Liposome |
| No. | HSPC | Chol. | PEG₂₀₀₀-DSPE | PEG₂₀₀₀-DS¹ | HSPG² | (mM) | Size |
| 16 | 281 (352) | 110 | 82.5 (30) | — | — | 41.4 | 124 nm ± 25 |
| | | (285) | | | | | |
| 17 | 281 (352) | 110 | — | 82.5 (31.1) | — | 37.3 | 111 nm ± 24 |
| | | (285) | | | | | |
| 18 | 281 (352) | 101.5 | — | — | — | 37.3 | 135 nm ± 3 |
| | | (263) | | | | | |
| 19 | 281 (352) | 101.5 | (30.4) | — | 24.38 (38) | 40.6 | 135 nm ± 29 |
| | | (263) | | | | | |

¹neutral lipopolymer prepared as described in Example 1
²negatively-charged, hydrogenated soy phosphatidylglycerol

Preparation nos. 16, 17, and 19 all included HSPC and cholesterol, but differed in the lipopolymer. Preparation no. 16 included PEG-DSPE, similar to preparation no. 3 described above. Preparation no. 17 included PEG-DS and preparation no. 19 included HSPG.

The liposome preparation nos. 16-19 and preparation no. 1 (Doxil®) were administered to pigs as described in Example 8. Typical hemodynamic changes were developed in about 3-6 minutes after the injection, including a 30-300% rise in pulmonary arterial pressure (PAP), variable rise and fall of systemic arterial blood pressure (SAP), tachycardia with or without subsequent bradyarrhythmia and decreases in Hb oxygen saturation. These changes were usually proportional with each other, although in some animals a propensity for cardiac vs. pulmonary response, manifested in severe bradyarrhytmia without major rises in PAP, was observed.

Table 7 summarizes the hemodynamic changes in the test animals. Twelve pigs numbered P1-P12 were used in this study, and the individual responses are indicated in Table 7. The changes in individual parameters were quantified as a percentage relative to preinjection baseline, and the overall response to each liposome preparation was arbitrarily qualified according to the Grade scoring system described in Example 7 (none (0), minimal (I), mild (II), severe (III), and lethal (IV)). Injection of 50-100 microliter from the preparation no. 1 (Doxil®) caused severe to lethal cardiopulmonary reaction in 9/9 pigs, whereas preparation no. 18 (HSPC/Chol vesicles) caused no reaction in all six pigs tested, even at 100-fold higher doses. Preparation no. 16 (HSPC/Chol/PEG-DSPE) caused mild to lethal reaction in ⅘ pigs, as did preparation no. 19 (HSPC/Chol/HSPG). Preparation no. 17, which included the neutral lipopolymer of the invention, (HSPC/Chol/PEG-DS) were resulted in mild reactions that were induced only at the highest dose level.

TABLE 7
Hemodynamic Response to Administration of Liposome Preparations

| | Reaction | | | | Individual Pig Responses | | | |
|---|---|---|---|---|---|---|---|---|
| Preparation | Overall | | Frequency | | Dose (mL/kg) | | | |
| No.¹ | Frequency | Grade² | n | % | 0.01-0.1 | 0.1-1 | 1-5 | 5-50 |
| 1 - Doxil ® | 9/9 | Severe | 3/9 | 33.3 | P1, P3, | P2, P9, | | |
| | | Lethal | 6/9 | 66.7 | P4, P5, | P4, P12 | | |
| | | | | | P6 | | | |
| 18 - | 0/6 | None | 6/6 | 100 | P7 | | P6, P8, | P9 |
| HSPC/Chol | | | | | | | P10, P11 | |
| 16 - | 4/5 | None | 1/5 | 20 | | | P9 | |
| HSPC/Chol/ | | Mild | 1/5 | 20 | | P7 | | |
| PEG-PE | | Lethal | 3/5 | 60 | | | P8, P10, | |
| | | | | | | | P11 | |
| 17 - | 3/4 | None | 1/4 | 25 | | | | P9 |
| HSPC/Chol/ | | Mild | 3/4 | 75 | | | P6, P10, | |
| PEG-DS | | | | | | | P11 | |
| 19 - | 5/5 | Mild | 3/5 | 60 | | | P6, P7, | |
| HSPC/Chol/ | | Lethal | 2/5 | 40 | | | P11 | P9, P10 |
| HSPG | | | | | | | | |

¹see Tables 6 and 3 for details of lipid composition of each preparation.
²see Example 7 for Grade scoring details.

In the studies described herein, liposome preparations with doxorubicin or cisplatin, or empty placebo liposomes, were selected as models for study. It will be appreciated that the findings that the neutral lipopolymer PEG-DS result in reduced induction of complement activation is applicable to liposomal preparations containing any entrapped drug or therapeutic agent. Exemplary agents include chemotherapeutic agents, antiviral agents, antibacterial agents, and the like. Doxorubicin, a chemotherapeutic agent, is an anthracycline antiobiotic, and other such compounds are contemplated, such as daunorubicin, epirubicin, and idarubicin. Cisplatin is also a platinum-containing chemotherapeutic agent, and other platium-containing drugs are contemplated, such as the varied cisplatin analogues known in the art, including but not limited to carboplatin, ormaplatin, oxaliplatin, ((−)-(R)-2-aminomethylpyrrolidine (1,1-cyclobutane dicarboxylato))platinum, zeniplatin, enloplatin, lobaplatin, (SP-4-3(R)-1,1-cyclobutane-dicarboxylato(2-)-(2-methyl-1,4-butanediamine-N,N′))platinum, nedaplatin, and bis-acetato-ammine-dichloro-cyclohexylamine-platinum(IV). It will be appreciated, however, that the findings herein are applicable to any drug or therapeutic agent.

### III. EXAMPLES

The following examples illustrate but are not intended in any way to limit the invention.

### Example 1A

### Synthesis of mPEG-DS (mPEG Aminopropanediol Distearoyl; α-methoxy-ω-2,3-di(stearoyloxy)propvlcarbamate Poly(ethylene Oxide))

A solution of mPEG₂₀₀₀ (20 g, 10 mol) was azeotropically dried in toluene (50 mL, 120° C.). After the temperature of the above solution reached 25° C., it was treated with nitrophenyl chloroformate (3.015 g, 15 mol) followed by TEA (2.01 mL, 15 mol). This mixture was allowed to react for 1½ hr. The TEA-salt was filtered and the solvent removed to give crude mPEG₂₀₀₀-nitrophenylchloroformate, to which a solution of aminopropanediol (3 g, 30 mol) in acetonitrile (50 mL) was added. This mixture was stirred overnight at room temperature. The insolubles were removed by filtration and the solvent was evaporated. The product was recrystallized twice from isopropanol. Yield: 13.7 g, 65%. ¹HNMR: (300 MHz, DMSO-D₆) δ 3.23 (s, OCH₃, 3H), 3.65 (s, PEG, 180H), 4.05 (t, urethane CH₂, 2H), 4.42 (t, 1° OH, 1H), 4.57 (d, 2° OH, 1H).

The product, mPEG₂₀₀₀ aminopropanediol (2.3 g, 1.08 mol, 2.17 meq of OH), was dissolved in toluene (30 mL) and azeotropically dried, removing about 10 mL of the solution. The solution was allowed to cool to room temperature. Pyridine (4 mL, 20%) was added by pipette, followed by addition of stearoyl chloride (1 g, 4.3 mol). Immediately a white precipitate was formed. The reaction mixture was refluxed overnight at 120° C. and allowed to cool. When the temperature of the reaction flask reached about 40° C., the pyridine salt was filtered. The filtrate was evaporated. The product (PEG₂₀₀₀-DS) was purified by recrystallizing twice from isopropanol (2×30 mL) and dried in vacuo over P₂O₅.

Yield: 2.26 g, 80%. TLC (chloroform:methanol, 90:10): mPEG aminopropanediol R_{f}=0.266; PEG-DS R_{f}=0.533. ¹HNMR: (300 MHz, DMSO-D₆) δ 0.89 (t, CH₃, 6H), 1.26 (s, CH₂, 56H), 1.50 (2t, 2CH₂, 4H), 2.24 (t, CH₂CH₂ C═O, 4H), 3.23 (s, OCH₃, 3H), 3.50 (s, PEG, 180H), 4.00 (dd, CH₂ of APD, 1H), 4.02 (t, CH₂OC═O—N, 2H), 4.20 (dd, CH₂ of APD, 1H), 4.98 (m, CHOC(O), 1H), 7.34 (m, NH, 1H).

A similar procedure was used to prepare mPEG-DS using mPEG polymers of molecular weight 750, 5000, and 12000. The structures were verified by ¹H-NMR and mass spectrometry. Molecular weights as determined by MALDI (Matrix Assisted Laser Desorption/lonization) are given below.

| Conjugate | MW by MALDI |
|---|---|
| mPEG(750)-DS | 1426 |
| mPEG(2000)-DS | 2892 |
| mPEG(5000)-DS | 5816 |
| mPEG(12000)-DS | 12729 |

### Example 1B

### Synthesis of PEG-DE (mPEG Aminopropanediol Diecosanoyl; α-methoxy-ω-2,3-di(ecosanovloxy)propylcarbamate Polv(ethylene Oxide))

In a 100 mL round bottom flask, ecosanoic acid (500 mg, 1.6 mmol) was dissolved in toluene (20 mL) and oxalyl chloride (147 μl, 1.68 mmol) was added by pipette. To the stirring reaction, 1% DMF was added. Upon addition of DMF, gas was released, as all contact with this gas should be avoided. After 10 minutes, the toluene was evaporated, and an additional 20 mL of toluene was added and evaporated to remove any excess of oxalyl chloride. The residue was redissolved in 10 mL of toluene. mPEG-aminopropanediol, prepared as described above, (1.19 g, 0.56 mmol) was added to the solution, a reflux condenser was attached, and the mixture was refluxed overnight. Analysis by TLC (methanol and chloroform, 9:1) showed the reaction to be complete. After the reaction mixture cooled, the undissolved material was filtered, and the filtrate was taken to dryness. The product was purified by recrystallizing three time from isopropanol and dried in vacuo over P₂O₅. Yield: 1.0 mg, 70%. ¹HNMR: (360 MHz, DMSO-D₆) δ 0.89 (t, CH₃, 6H), 1.26 (s, CH₂, 66H of lipid), 1.50 (t, 2CH₂, 4H), 2.24 (t, CH₂CH₂ C═O, 4H), 3.23 (s, OCH₃, 3H), 3.50 (s, PEG, 180H), 4.00 (dd, CH₂ of APD, 1H), 4.05 (t, CH₂^{j}CH₂C+O, 4H), 3.23 (s, OCH₃, 3H, 3.50 (s, PEG, 180H), 4.00 (dd, CH₂ of APD, 1H), 4.05 (t, CH₂OC═O—N, 2H), 4.20 (dd, CH₂ of APD, 1H), 4.98 (m, CHOC(O), 1H), 7.34 (m, NH,1 H) ppm.

### Example 2

### Preparation of t-Bu-O-PEG-Aminopropanediol via t-Bu-O-PEG-O-Succinimide

A. t-Bu-O-PEG-O-Succinimide

tBu-O-PEG-2000 from Polymer Labs (10 g, 5 mmol) was azeotropically dried by dissolving in 120 mL toluene and removing about 20 mL of the solvent, collecting any water in a Dean Stark trap.

The solution was cooled to room temperature, and phosgene (15 mL) was added. The mixture was allowed to react overnight at room temperature. After the completion of the reaction, the solvent was removed by rotary evaporator. About 50 mL of fresh toluene was added and removed by rotary evaporator. The residue was dissolved in dry toluene (30 mL) and methylene chloride (10 mL). To this solution, N-hydroxysuccinimide (1.7 g,14.8 mmol) and triethylamine (2.1 mL, 14.9 mmol) were added, and the mixture was allowed to react overnight at room temperature, after which time the reaction was complete by TLC.

| Compound | R_{f} (CHCl₃:CH₃OH, 90:10) |
|---|---|
| t-Bu-O-PEG-OH | 0.44 |
| t-Bu-O-PEG-OSc | 0.51 |

The salt was filtered from the reaction mixture, the solvent was removed by evaporation, and the solid was recrystallized twice from isopropyl alcohol and dried over P₂O₅. Yield: 9.2, 85%. ¹HNMR: (CDCl₃, 360 MHz) δ 1.25 (s, t-Bu, 9H), 2.82 (s, CH₂CH₂, 4H), 3.60 (s, PEG, 180H), 4.45 (t, CH₂OCONH, 2H) ppm.

B. t-Bu-O-PEG-Aminopropanediol

To a solution of aminopropanediol (300 mg, 3.2 mmol) in DMF (10 mL), t-Bu-PEG-OSc (5 g, 2.29 mmol) was added and allowed to react overnight. All NHS ester was consumed, giving a mixture showing one spot on TLC.

| | R_{f} (CHCl₃:CH₃OH, |
|---|---|
| Compound | 90:10) |
| t-Bu-O-PEG-OSc | 0.51 |
| t-Bu-O-PEG-APD | 0.35 |

A previously washed acidic ion exchange resin (˜1 g) was added to the reaction mixture and removed by filtration after 30 minutes. The solvent was removed and the residue recrystallized from 200 mL of isopropyl alcohol. The solid was collected and dried over P₂O₅. Yield: 4.2 g, 85%. ¹HNMR: (D6-DMSO, 360 MHz) δ 1.25 (s, t-Bu, 9H), 3.68 (s, PEG, 180H), 4.03 (t, CH₂OCONH, 2H), 4.43 (t, 1° OH, 1H), 4.55 (d, 2° OH, 1H), 6.98 (t, NH, 1H) ppm.

### Example 3

### Preparation of p-Nitrophenylcarbonate-PEG-DS

A. Bn-O-PEG-Nitrophenylcarbonate (NPC)

Bn-O-PEG-2000 from Shearwater Polymers (Huntsville, La.; 5 g, 2.41 mmol) was azeotropically dried by dissolving in 120 mL toluene and removing about 20 mL of the solvent, collecting any water in a Dean Stark trap. The solution was cooled to room temperature and remaining solvent was evaporated under reduced pressure.

The residue was dissolved in 30 mL of methylene chloride/ethyl acetate (60:40), and p-nitrophenylchloroformate (729 mg, 3.6 mmol) and triethylamine (1 mL, 7.2 mmol) were added. The reaction was carried out at 4° C. for 8-16 hours. This method slows down the reaction but eliminates the formation of bis PEG-carbonate. A UV visible spot on GF silica plate indicated the completion of the reaction.

The reaction mixture was treated with previously cleaned acidic and basic ion exchange resin for 30 minutes, filtered, and taken to complete dryness. The product was recrystallized from isopropyl alcohol and dried over P₂O₅. Yield: 4.4 g, 80%.

B. Bn-O-PEG-Aminopropanediol

To a solution of aminopropanediol (260 mg, 1.9 mmol) in DMF (10 mL), Bn-O-PEG-NPC, as prepared above (4.3 g, 2.9 mmol), was added and reacted for 5 hours. All Bn-O-PEG-NPC was consumed, the reaction mixture giving one spot on TLC (chloroform:methanol:water 90:18:2).

The reaction mixture was treated with 5 g previously cleaned acidic ion exchange resin for 30 minutes, filtered, and taken to complete dryness. The product was recrystallized from isopropyl alcohol and dried over P₂O₅. Yield: 3.8 g, 91%.

C. Bn-O-PEG-Distearoyl

A solution of Bn-O-PEG-aminopropanediol (3 g, 1.36 mmol), stearic acid (1.94 g, 6.79 mmol), and DPTS (4-(dimethylamino)pyridinium 4-toluenesulfonate) as catalyst (408 mg, 1.36 mmol) was stirred at room temperature for 20 minutes. Diisopropylcarbodiimide (1.28 mL, 8.16 mmol) was added by pipette and the mixture allowed to react overnight. TLC (chloroform:methanol, 90:10) showed complete reaction of the diol.

Basic ion exchange resin (˜5g) was added to the reaction mixture. After 30 minutes of shaking, the resin was filtered and the filtrate was taken to dryness. The residue was recrystallized from isopropanol (100 mL) and dried over P₂O₅. Yield: 4 g, 80%.

D. HO-PEG-Distearoyl

Two different approaches were taken for the deprotection of the benzyl group of Bn-O-PEG-DS.

Method 1. Hydrogenolysis: Deprotection by Palladium on Carbon. To a solution of Bn-O-PEG-DS (218 mg, 0.08 mmol) in 5 mL of methanol, 10% Pd/C (110 mg) and ammonium formate (107 mg, 0.8 mmol) were added and the mixture allowed to reacted at room temperature overnight.

Pd/C was removed by filtration over Celite®, and the filtrate was taken to dryness. The residue was dissolved in chloroform and washed three times with saturated NaCl. The chloroform phase was collected, dried with MgSO₄, filtered and concentrated. The solid residue was lyophilized from tBuOH, and the resulting powder was dried over P₂O₅. Yield: 80%, 175 mg.

Method 2. Deprotection by Titanium Tetrachloride. A solution of Bn-O-PEG-DS (1.18 g, 0.43 mmol) in methylene chloride (10 mL) was cooled in an ice bath for 5 minutes. Titanium tetrachloride (3 mL, 21.5 mol, excess) was transferred via an oven dried syringe into the sealed reaction flask. After 5 minutes, the ice bath was removed, and the deprotection reaction was carried out overnight at room temperature. Complete deprotection was shown by a lower shifted spot (relative to starting material) on a GF silica TLC plate.

About 40 mL of chloroform was added to the reaction mixture, and the mixture was transferred to a separatory funnel containing 40 mL of saturated NaHCO₃. The mixture was shaken gently (to avoid formation of an emulsion) and the chloroform layer was collected. This extraction was repeated 3 times, and the chloroform phase was collected and was extracted once more with a fresh portion of saturated NaHCO₃ to ensure complete removal of TiCl₄. The collected chloroform phase was dried with MgSO₄, filtered and concentrated.

The above residue was dissolved in 1 mL of chloroform and added to a prepared column of silica gel (200-400 mesh, 60 Å). The polarity of the mobile phase (chloroform) was increased by 2% incremental additions of methanol until the product eluted at 10% methanol/90% chloroform. The product was collected and the solvent removed by rotary evaporator. The solid was lyophilized from tBuOH and dried over P₂O₅. Yield: 70%, 800 mg.

E. p-Nitrophenylcarbonate-PEG-DS

The reaction flask, stirring bar, syringes and starting material (HO-PEG-DS, as prepared above) were meticulously dried before start of the reaction.

To a solution of HO-PEG-DS (1.2 g, 0.45 mmol) in 10 mL of methylene chloride/ethyl acetate (60:40), p-nitrophenylcarbonate (136 mg, 0.65 mmol) and triethylamine (188 μL, 1.35 mmol) were added. The reaction was carried out at 4° C. (to eliminate the formation of bisPEG-carbonate) for 8-16 hours, after which time the reaction was complete by GF silica gel TLC.

| Compound | R_{f} (CHCl₃:CH₃OH, 90:10) |
|---|---|
| HO-PEG-DS | 0.40 |
| NPC-PEG-DS | 0.54 |

The reaction mixture was treated for 30 minutes with previously cleaned acidic and basic ion exchange resins and filtered. The filtrate was taken to complete dryness and the residue recrystallized from isopropyl alcohol. The solid was dried over P₂O₅. Yield: 70%. ¹NHMR: (D6-DMSO, 360 MHz) δ 0.86 (t, CH₃, 6H), 1.22 (s, DS, 56H), 1.48 (m, CH₂CH₂(CO)), 4H), 2.26 (2xt, CH₂OCONH, 2H), 4.03 & 4.22 (2xd, CH₂CH of lipid, 2H), 4.97 (M, CHCH₂ of lipid), 6.98 (t, NH, 1H), 7.55% 8.32 (2xd, aromatic, 4H) ppm.

### Example 4

### Preparation of Neutral-zwitterionic mPEG-DSPE by Reductive Amination Coupling of mPEG-NH₂ and Periodate-oxidized DSPG

1,2-Distearoyl-sn-glycero-3-phospho-rac[(1-glycerol)] or distearoyl phosphatidylglycerol (DSPG, 200 mg, 0.25 mmol) was suspended in sodium acetate saline buffer (1.5 mL, 50 mM, pH=5) and treated with sodium periodate (348 mg, 1.6 mmol) for 4 h while the suspention was sonicated. TLC (chloroform:methanol:water=90:18:2) showed that DSPG was consumed. The insoluble product was separated from the solution after centrifugation and then washed with water (1 mL), water/acetonitrile, 1:1 (2 mL, twice), and then with acetonitrile only (1 mL, 3 times). The product was dried in vacuo over P₂O₅ for 1.5 h mPEG-NH₂ (1 g, 0.5 mmol, 2 eq) was added to the oxidized DSPG with benzene (3 ml), and the solvent was rotary evaporated to remove the remaining water. The benzene evaporation step was repeated 2 more times. Dry methanol (6 mL) and powdered molecular seives (4 Å, 320 mg) were added to the mixture followed by borant-pyridine (8M, 1.6 mL, 12 mmol). The reaction mixture was stirred at 25° C. for 15 h. TLC confirmed formation of the lipopolymer product. In order to remove the excess of unreacted mPEG-NH₂ the product mixture was diluted with water (3 mL), transferred to spectropore CE dialysis membrane (MWCO 300,000), and dialyzed at 4° C. against saline solution (˜50 mM, 1000 mL, 3 times), and then against deionized water (3 times). The crude product (by TLC, contaminated with some oxidized DSPG) was lyophilized and dried in vacuo over P₂O₅ and further purified by silica gel column chromatography using methanol gradient (0-15%) in chloroform as eluent. The fractions containg the pure lipopolymer product were pooled, and evaporated to yield 141 mg (20%) solid. ¹H NMR (360 MHz, CDCl₃) δ: 0.88 (t, CH₃, 6H); 1.26 (s, CH₂, 56H); 1.58 (m, CH₂CH₂CO, 4H); 2.28 (2xt, CH₂CO, 4H); 3.2 (br m, NHCH₂CH₂, 1H); 3.32 (br m, NHCH₂CH₂, 1H); 3.6 (s, PEG˜180H); 4.15 (dd, trans PO₄CH₂CH, 1H); 4.35 (dd, cis PO₄CH₂CH, 1H); 5.2 (m, PO₄CH₂CH, 1H). MALDI-TOFMS produced a bell-shaped distribution of ions spaced at equal 44 Dalton intervals and centered at 2770 Daltons (calculated molecular weight: 2813 Daltons).

### Example 5

### Preparation and Biodistribution Studies of PEG-DSPE- and PEG-DS-Containing Liposomes

Lipid films were formed, by dissolution and removal of solvent, from mixtures of HSPC:Chol:PEG-lipid in the following ratios:

A: 58:39:3; PEG-lipid=PEG-DS

B: 57:38:5; PEG-lipid=PEG-DSPE

C: 57:38:5; PEG-lipid=PEG-DS

The films were hydrated in freshly prepared ¹²⁵I-Tyraminylinulin in 25 mM HEPES containing 140 mM NaCl, pH 7.4, and extruded to form liposomes 100-105 nm in diameter. The liposomes were sterilized by filtration through 0.22 μm Millipore (Millipore Corporation, Bedford, Mass.) low protein-binding syringe-end filters. Aliquots were counted to determine the injection counts of ¹²⁵I. Lipid concentrations were determined by assaying the phosphate content of the liposome preparations, and the liposome preparations were diluted in sterile buffer to a final concentration of 2.5 μmol/mL. Mice were injected i.v. via the tail vein with 0.2 mL of the diluted liposomes, so that each mouse received 0.5 μmol of phospholipid. At the various time points, mice were euthanised by halothane anesthesia followed by cervical dislocation, the blood sampled by cardiac bleeds, and the blood and various organs assayed for ¹²⁵I counts.

### Example 6

### Measurement of Complement Activation In Vitro

Materials

Dimyristoyl phosphatidylcholine (DMPC), dimyristoyl phosphatidyl-glycerol (DMPG), cholesterol (Chol) and egg yolk lecithin (EPC) were purchased from Avanti Polar Lipids (Alabaster, Ala.), and fully hydrogenated soy phosphatidylcholine (HSPC) and the fully hydrogenated soy phosphatidylglycerol (HSPG) were from Lipoid Inc., Ludwigshafen, Germany. All lipids had a purity of ≧97%. Zymosan was from Sigma Chem. Co. (St. Louis, Mo.).

Commercial Doxil® was obtained from ALZA Corp (Mountain View, Calif.) and contained doxorubicin HCl, 2 mg/mL (4.22 mM), liposomal lipid, 16 mg/mL, ammonium sulfate, ≈0.2 mg/mL; histidine, 10 mM (pH 6.5) and sucrose, 10%. The lipid constituents included HSPC, 9.58 mg/mL; Chol, 3.19 mg/mL; PEG₂₀₀₀-DSPE, 3.19 mg/mL (total phospholipid, 12.8 mg/mL, 13.3 mM).

N-carbamyl-poly(ethylene glycol methyl ether)-1,2-distearoyl-sn-glycerol-3-phosphoethanol-amine triethyl ammonium salt (PEG-DSPE) having a PEG moiety of 350 Daltons, 2000 Daltons, and 12,000 Daltons (PEG₃₅₀-DSPE; PEG₂₀₀₀-DSPE and PEG₁₂₀₀₀-DSPE, also referred to as 0.35 K-PEG-DSPE; 2.0 K PEG-DSPE; 12.0 K PEG-DSPE, respectively) were obtained from Alza Corporation.

3-methoxy polyethylene glycol-oxycarbonyl 3-amino-1,2 propandiol distearoyl ester having polyethyleneglycol (PEG of moiety of 2000 Da (PEG₂₀₀₀-DS, also referred to as 2K-PEG-DS) was prepared as described above.

3-methoxy-polyethelene glycol 1,2 distearoyl glycerol (PEG₂₀₀₀-DSG, also referred to as 2K-PEG-DSG) (Sunbright DSG-2H) was obtained from Nippon Oil & Fat Co., Ltd (Tokyo, Japan).

Human serum was obtained from healthy volunteer donors. The sera were kept at −70° C. until use.

Methods

Determination of phospholipid concentration: Phospholipid concentration was determined using a modification of the Bartlett procedure.

Particle size distribution determination: Particle size distribution was determined by dynamic light scattering at 25° C. using either High Performance Particle Sizer ALV-NIBS/HPPS with ALV-5000/EPP multiply digital correlator (ALV-Laser Vertriebsgesellschaft GmbH, Langen, Germany), or a Nicomp Model 370 (Pacific Scientific, Silver Spring, Md.) submicron particle sizer.

Measurement of liposome surface charge (Ψ potential): To determine electrical surface potential of liposomes, the degree of HC ionization over a broad range of pH values was measured. An aliquot of 30 μL of liposomes was diluted in 1.5 mL of buffers. pH was adjusted by addition of an appropriate amount of concentrated sodium hydroxide and hydrochloric acid. All samples were sonicated for about 5 seconds in a water bath to ensure pH equilibrium between the inside and the outside of the large unilamellar vesicle (LUV). To measure the HC ionization state, HC fluorescence excitation spectra were recorded at room temperature (22° C.) using an LS550B luminescence spectrometer (Perkin Elmer, Norwalk, Conn.). Measurements were carried out at two excitation wavelengths: 330 nm, which is pH independent (isosbestic point) and represents the total amount of HC (un-ionized+ionized) in the lipid environment, and 380 nm, which reflects only the ionized HC⁻. The emission wavelength was 450 nm for both excitation wavelengths. Excitation and emission bandwidths of 2.5 nm were used. For each lipid composition, the apparent pKa of HC was calculated from the change of the ratio of excitation wavelengths 380/330 as a function of bulk pH. A shift in the apparent pKa of HC, which represents its apparent proton binding constant, relative to a reference neutral surface, is indicative of the surface pH and the electrical surface potential in the immediate environment of the HC fluorophore. The values for electrical surface potential (Ψ) was calculated using the equation:

$${Ψ}_{0} = - \frac{Δ {pK}_{el} kT}{ⅇ l n 10}$$

Determination of Zeta Potential: Zeta potential was measured at 25° C. using a Zetasizer 3000 HAS, Malvern Instruments Ltd, Malvern, UK. An aliquot of 40 μL of liposomes was diluted in 20 mL of 10 mM NaCl (pH 6.7) and the solutions were passed through a 0.2-μm syringe filter (Minisart, Sartorius, Germany). The principle of measurement is the following: when an electrical field is applied to a suspension of charged particles in an electrolyte, the velocity of their movement towards the electrode of opposite polarity depends on the strength of the field, the dielectric constant, the viscosity of the medium, and the zeta-potential. The relationship of zeta potential to the particle velocity in a unit electric field (electrophoretic mobility) is described by the Henry equation:

$${U}_{E} = \frac{zϵf ( Ka )}{6 π η}$$

where U_{E}=electrophoretic mobility, z=zeta potential, ε=dielectric constant, and η=viscosity. f(Kₐ) is a function of the electric double layer thickness and particle diameter. In aqueous media or moderate electrolyte concentrations (10 mM NaCl), f(Ka) value is 1.5, which is used in the Smoluchowski approximation:

$${U}_{E} = \frac{ϵ z}{4 π n}$$

At 25° C., the zeta potential can be approximated as:

z=12.85 U_{E} mV

A. Liposome Preparation

Liposomes comprised of the various lipid compositions shown in Table 3 were prepared as follows. The lipid components of each formulation were dissolved in tertiary butanol. The clear solution was freeze-dried. The powder was hydrated in 10 mL hot (65° C.) sterile pyrogen-free saline by vortexing for 2-3 min at 70° C. to form multilamellar vesicles (MLV). The MLVs were downsized in two extrusion steps through polycarbonate filters of 0.4 and 0.1 μm pore size, 10 times through each, using TEX 020 10 mL barrel extruder from Northern Lipids Inc. (formerly Lipex, Vancouver, BC, Canada), at 62° C. All steps of liposome preparation were done aseptically. Liposomes were suspended in 0.15 M NaCl/5 mM histidine buffer (pH 6.5). All liposome preparations were sterile and pyrogen free.

Micelles were prepared by extensive vortex mixing of 2K-PEG-DSPE or 2K-PEG-DS in saline at 2 mg/mL followed by filtration through 0.22 μm filters.

TABLE 3
Liposome Compositions

| Formulation Number and | | |
|---|---|---|
| Name | Lipid Composition (+drug) | Lipid Molar Ratio |
| 1 - Doxil ® | HSPC/Chol/PEG₂₀₀₀-DSPE (+doxorubicin) | 56:38.6:5.4 |
| 2 - cisplatin liposomes | HSPC/Chol/PEG₂₀₀₀-DSPE (+cisplatin) | 56:38.6:5.4 |
| 3 - Doxil ® placebo | HSPC/Chol/PEG₂₀₀₀-DSPE | 56:38.6:5.4 |
| 4 - 0.6% PEG₂₀₀₀-DSPE | HSPC/Chol/PEG₂₀₀₀-DSPE | 54.7:44.6:0.62 |
| 5 - HSPC/Chol | HSPC/Chol | 57.2:42.8 |
| 6 - PEG-DS | HSPC/Chol/PEG₂₀₀₀-DS | 54.3:42.7:4.5 |
| 7 - EPC/PEG-DSG | EPC/PEG₂₀₀₀-DSG | 95.5:4.5 |
| 8 - PEG₃₅₀-DSPE | HSPC/Chol/PEG₃₅₀-DSPE | 54.3:41.3:4.3 |
| 9 - PEG₁₂₀₀₀-DSPE | HSPC/Chol/PEG₁₂₀₀₀-DSPE | 54.3:41.3:4.3 |
| 10 - HSPG | HSPC/Chol/HSPG | 38.1:28.4:33.5 |
| 11 - Low-Chol | DMPC/Chol/DMPG | 45:50:4 |
| 12 - High-Chol | DMPC/Chol/DMPG | 24:71:5 |
| 13 - PEG-DSPE micelles | PEG₂₀₀₀-DSPE micelles | 100 |
| 14 - PEG-DS micelles | PEG₂₀₀₀-DS micelles | 100 |

B. In vitro Complement Activation Measurement

Liposomes were incubated with undiluted human serum in a shaking water bath (80 cycle/min) and complement activation was estimated by measuring the formation of complement terminal complex SC5b-9. In a typical experiment 10 μL liposomes was mixed with 40 μL serum in Eppendorf tubes which were then incubated for 30 minutes at 37° C. in a shaking water bath (shaking rate of 80 rpm). The reaction was stopped by adding 20 volumes of 10 mM EDTA, 25 mg/mL bovine serum albumin, 0.05% Tween 20 and 0.01% thimerosal (pH 7.4) (i.e., the “sample diluent” of the SC5b-9 ELISA kit supplemented with EDTA). SC5b-9 was determined by an enzyme-linked immunosorbent assay (Quidel Co., San Diego, Calif.), as previously described (Szebeni, J. et al. J. Natl. Cancer Inst., 90:300 (1998)).

### Example 7

### Measurement of Complement Activation In Vivo

Liposomes prepared as described in Example 6 were administered to pigs as follows. Yorkshire swine of both sexes in the 25-40 kg range were obtained. Animals were sedated with i.m. ketamine (500 mg) and anesthetized with 2% isoflurane, using an anesthesia machine. A pulmonary artery catheter was advanced via the right internal jugular vein through the right atrium into the pulmonary artery to measure pulmonary artery wedge pressure (PAP). Systemic arterial pressure (SAP) was measured in the femoral artery. Other details of surgery, instrumentation, and hemodynamic analysis were performed as described previously (Szebeni, J. et al., Circulation, 99:2302 (1999)).

The indicated amounts of each liposome preparation was diluted in 1 mL PBS and injected into the jugular vein, via the catheter introducer, or directly into the pulmonary artery, via the pulmonary arterial catheter. These injection methods were equivalent in inducing hemodynamic changes. Liposomes were flushed into the circulation with 5-10-mL PBS. To provide a composite measure of liposome reactions, the hemodynamic changes were quantified by an arbitrary grading scheme by monitoring for one of the following physiological abnormalities:

### Abnormality

Rise of PAP

Rise or fall of systemic arterial pressure (SAP)

Fall of cardiac output

EKG abnormalities

Fall of exhaled CO2

Rise or fall of heart rate

Rise of plasma TXB2

Rise of pulmonary and systemic vascular resistance

Flushing

The liposome-induced cardiovascular reactions in pigs was scaled as follows:

| Grade | Symptoms |
|---|---|
| 0 (none) | no significant alteration in ECG or any hemodynamic parameters |
| I (minimal) | transient (<2 min), <20%, clearly distinguishable changes in one or more of the |
| | following parameters: heart rate, ECG, SAP, PAP, Hb oxygen saturation |
| II (mild) | transient (<2 min), >20% but <50% changes in one or more of the following |
| | parameters: heart rate, ECG, SAP, PAP, Hb oxygen saturation |
| III (severe) | more extended (up to 10 min) >50 changes in more than one of the above |
| | parameters, +bradyarrhythmia |
| IV (lethal) | Lethal reaction: circulatory collapse within 4 min requiring epinephrine and/or |
| | CPR with defibrillation. Typically SAP falls to <40 mm Hg, PAP rises to |
| | maximum (cc 60 mmHg), tachycardia is followed by severe bradycardia with |
| | arrhythmia, leading to cardiac arrest and death |

### Example 8

### In vivo Characterization of Liposome Preparations

Four liposome (LUV) preparations were made. The liposome compositions and characterizations are set forth in Table 6. In preparing each of the formulations all lipid components of the formulation were dissolved in tertiary butanol. The clear solution was freeze-dried. The powder was hydrated in 10 mL hot (65° C.) sterile pyrogen-free saline by vortexing for 1 minute at 70° C. to form MLV. The MLV were downsized in two extrusion steps through polycarbonate filters of 0.4 and 0.4 micron pore size 10 times through each using TEX 020 10 ml barrel extruded from Northern Lipids (previously Lipex), Vancouver, BC, Canada at 62° C. All steps of liposome preparation were done aseptically.

Commercial Doxil® was used (phospholipid concentration 13.3 mM, 150 μg doxorubicin/μmol phospholipid). All other liposomes were prepared in saline (0.9% NaCl) and lack a (NH₄SO₄) gradient. All liposomes used were in the size range 105 nm±35 nm (see Table 6).

The indicated amounts of Doxil® and other test liposomes were diluted in 1 mL PBS and injected into the right ventricle, or directly into the pulmonary artery of pigs, via the pulmonary arterial catheter. Liposomes were flushed into the circulation with 10 mL PBS. Previous findings indicated that the hemodynamic effects of small liposome boluses were nontachyphylactic and quantitatively reproducible several times in the same animal, therefor increasing amounts of the same type of liposomes were injected in each pig until a reaction developed, or, in the absence of reaction, until a certain predetermined top dose was tested. The results are shown in Table 7.

While the invention has been described with reference to specific methods and embodiments, it will be appreciated that various modifications may be made without departing from the invention.

It is claimed:

1. A method of reducing liposome-induced complement activation upon in vivo administration of liposomes containing an entrapped therapeutic agent, comprising
providing liposomes comprised of a vesicle-forming lipid and between 1-10 mole percent of a neutral lipopolymer having the formula: 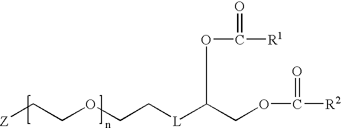
where each of R¹ and R² is an alkyl or alkenyl chain having between 8 and 24 carbon atoms;
n=10-300,
Z is selected from the group consisting of C₁-C₃ alkoxy, C₁-C₃ alkyl ether, n-methylamide, dimethylamide, methylcarbonate, dimethylcarbonate, carbamate, amide, n-methylacetamide, hydroxy, benzyloxy, carboxylic ester, C₁-C₃ alkyl carbonate, and aryl carbonate; and
L is selected from the group consisting of (i) —X—(C═O)—Y—CH₂—, (ii) —X—(C═O)—, and (iii) —X—CH₂—, where X and Y are independently selected from oxygen, NH, and a direct bond, with the proviso that when L is —X—(C═O)—, X is not NH;
and the remainder vesicle-forming lipids.

2. The method of claim 1, wherein X is oxygen and Y is nitrogen.

3. The method of claim 1, wherein L is a carbamate linkage, an ester linkage, or a carbonate linkage.

4. The method of claim 3, wherein L is —O—(C═O)—NH—CH₂— (a carbamate linkage).

5. The method of claim 1, wherein Z is hydroxy or methoxy.

6. The method of claim 1, wherein said preparing includes preparing liposomes containing about 1 to 10 mole % of the neutral lipopolymer distearoyl (carbamate-linked) polyethylene glycol.

7. The method of claim 1, wherein said preparing includes preparing liposomes containing about 1 to 10 mole % of the neutral lipopolymer methoxy-polyethelene glycol 1,2 distearoyl glycerol.

8. The method of claim 1, wherein each of R¹ and R² is an unbranched alkyl or alkenyl chain having between 8 and 24 carbon atoms.

9. The method of claim 8 wherein each of R¹ and R² is C₁₇H₃₅.

10. The method of claim 1, wherein n is between about 20 and about 115.

11. The method of claim 1, wherein the therapeutic drug is a chemotherapeutic agent.

12. The method of claim 11, wherein said chemotherapeutic agent is an anthracycline antiobiotic.

13. The method of claim 12, wherein said chemotherapeutic agent selected from the group consisting of doxorubicin, daunorubicin, epirubicin, and idarubicin.

14. The method of claim 11, wherein said chemotherapeutic agent is a platinum-containing compound.

15. The method of claim 14, wherein said platinum-containing antibiotic is cisplatin or a cisplatin analogue selected from the group consisting of carboplatin, ormaplatin, oxaliplatin, ((−)-(R)-2-aminomethylpyrrolidine (1,1-cyclobutane dicarboxylato))platinum, zeniplatin, enloplatin, lobaplatin, (SP-4-3(R)-1,1-cyclobutane-dicarboxylato(2-)-(2-methyl-1,4-butanediamine-N,N′))platinum, nedaplatin and bis-acetato-ammine-dichloro-cyclohexylamine-platinum(IV).
